(19) United States
(12) Patent Application Publication
(10) Pub. No.: US 2003/0171345 A1
(43) Pub. Date: Sep. 11, 2003

(54) 8Beta-substituted 11beta-aryl-estra-1,3,5,(10)-triene derivatives

(75) Inventors: Nico Brauer, Jena (DE); Olaf Peters, Jena (DE); Alexander Hillisch, Jena (DE); Christa Hegele-Hartung, Mulheim a.d Ruhr (DE); Peter Muhn, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/270,077

(22) Filed: Oct. 15, 2002

Related U.S. Application Data
(60) Provisional application No. 60/330,728, filed on Oct. 29, 2001.

(30) Foreign Application Priority Data
Oct. 15, 2001 (DE) 101 51 114.0

(51) Int. Cl. C07J 43/00; A61K 31/58; A61K 31/56; C07J 1/00

(52) U.S. Cl. 514/176; 514/179; 540/107; 552/623

(57) ABSTRACT

This invention relates to 11β-(para-substituted)phenyl-estra-1,3,5(10)-trienes with a straight-chin or branched-chain, optionally partially or completely halogenated alkyl radical or alkenyl radical in each case with up to 5 carbon atoms, an ethinyl radical or prop-1-inyl radical in 8β-position.

The new compounds can be used for contraception in men and women, without influencing other estrogen-sensitive organs, such as the uterus or the liver. They are also suitable for the treatment of benign or malignant proliferative diseases of the ovary, such as ovarian cancer and granulosa cell tumors.

This application claims the benefit of the filing date of U.S. Provisional Application Serial No. 60/330,728 filed Oct. 29, 2001.

### FIELD OF THE INVENTION

This invention relates to new compounds as pharmaceutical active ingredients that have in vitro a higher affinity to estrogen receptor preparations from rat prostates than to estrogen receptor preparations from rat uteri and exert in vivo a contraceptive action by their preferential action on the ovary, their production, their therapeutic use and pharmaceutical dispensing forms that contain the new compounds.

The chemical compounds are novel, steroidal, tissue-selective estrogens.

### BACKGROUND OF THE INVENTION

Contraceptive methods with chemical compounds are common with women who do not want to become pregnant. The following chemical methods of female contraception are now available to us:

The endocrine principle: suppression of ovulation by inhibition of the release of gonadotrophin and thus the ovulation

Prevention of the ascension of sperm through the female reproductive tract to the fallopian tube where the fertilization takes place

Prevention of the implantation or nidation of a fertilized embryo in the uterus

Spermicide

Abortion-inducing agent

Oral contraceptives that consist of the most varied combinations of an estrogen with a gestagen are the most frequently used contraceptives of women. They act according to the endocrine principle. Although such contraceptives are very effective, undesirable side effects may occur, however, such as, e.g., irregular bleeding, nausea, vomiting, depression, weight gain or headaches. More serious diseases are also sometimes observed, such as thrombo-embolisms, stroke, liver adenoma, gallbladder diseases or hypertension, which indicate that no effective contraceptives without side effects are now available. The medical necessity for a new contraceptive method thus exists.

An ideal contraceptive method is a method that operates directly on the ovarian follicle without influencing the endocrine hypothalamo-pituitary-ovarian axis. This can be achieved with a chemical compound that impairs the folliculogenesis, for example by destroying a paracrine interaction between the egg cell and the granulosa cells, and thus provides that

the follicle program cannot proceed adequately, so that an incompetent egg cell matures, which is ovulated but cannot be fertilized, or

the follicle program cannot proceed adequately, so that an incompetent egg cell matures, which is ovulated and fertilized but does not result in any pre-implantation development, or

the folliculogenesis is possible only to a limited extent, and it does not result in any ovulation.

Follicular growth is the development of an ovarian follicle from the primordial stage to the large antral follicle that is ready to burst. Only an optimally built-up antral follicle has the potential to ovulate a mature egg cell. Patients with ovarian infertility, e.g., PCOS (=polycystic ovarian syndrome) patients, have a disrupted folliculogenesis associated with hormonal and ovulation disorders as well as insufficiently matured egg cells (Franks et al. (2000) Mol Cell Endocrinol 163: 49-52).

There are always more indications that the early stages of folliculogenesis, i.e., the development steps from the primordial follicle to the early antral follicle, are gonadotrophin-independent, but it is still not conclusively explained which of the identified autocrine or paracrine factors (Elvin et al. (1999), Mol Cell Endocrinol 13: 1035-1048; McNatty et al. (1999), J Reprod Fertil Suppl 54: 3-16) are the most important in early folliculogenesis. Gonadotrophins, such as, e.g., FSH (follicle-stimulating hormone), however, are mainly involved in the late steps of folliculogenesis, i.e., the development from the early antral follicle to the large ovulatory follicle. Additional modulators of folliculogenesis are also discussed in the late folliculogenesis, however (Elvin et al. (1999), Mol Cell Endocrinol 13: 1035-1048).

Estrogen receptor β (ERβ) was recently discovered as a second subtype of the estrogen receptor (Kuiper et al. (1996), Proc. Natl. Acad. Sci. 93: 5925-5930; Mosselman, Dijkema (1996) Febs Letters 392: 49-53; Tremblay et al. (1997), Molecular Endocrinology 11: 353-365). The expression pattern of ERβ differs from that of the ERα. (Kuiper et al. (1 996), Endocrinology 13 8: 863-870). Whereas an expression of ERα was detectable in almost all organs studied, the highest expression of ERβ in female animals was found in the ovary and in male animals was found in the prostate (Couse et al. (1997) Endocrinology 138: 4613-4621). In the ovary, a clear ERβ expression in follicles is shown in almost all stages of development: While in the follicles ERα is expressed only in the outside follicle cells (thecal cells), a strong expression of ERβ is present in the estradiol-producing granulosa cells. Based on the varying cell distribution of ERα and ERβ in the ovarian follicle, it is thus to be expected that the interaction of a ligand with ERα or ERβ will lead to different cellular responses. The fact that ERα and ERβ are functionally different was recently confirmed by the successful production of ERα and ERβ knockout mice (Couse et al. (1999), Endocrine Reviews 20: 358-417). ERα is consequently decisively involved in the function of the uterus, the mammary gland, the control of the sexual-endocrine axis, whereas ERβ is included predominantly in the processes of ovarian physiology, especially folliculogenesis and ovulation.

Another organ system with high ERβ expression is the testis (Mosselmann et al. 1996 Febs Lett 392 49-53) including the spermatides (Shugrue et al. 1998, Steroids 63: 498-504). The fact that ERβ is functional in the male animal also arises through studies of ERα-(ERKO) or ERβ-(βERKO)-knockout mice: Male ERKO mice (Hess, R. A. et al. 1997, Nature 390: 509-512) have considerable fertility disorders. As a result, the important function of estrogens with respect to maintaining testis function relative to fertility is confirmed.

ERα and ERβ have significantly different amino acid sequences in their ligand binding domains and transactivation domains. This suggests that (1) ER subtypes bind to their ligands with different affinity and (2) ligands can show a different agonistic and/or antagonistic potential on the two receptor subtypes.

Patent Applications WO 00/47603, WO 00/63228, PCT/EP00/10804, DE 100 19167.3, U.S. Ser. No. 60/207,370 as well as publications (Sun et al. (1999), Endocrinology 140: 800-804; Stauffer et al: (2000), J Comb Chem 2: 318-329) recently showed that steroidal and nonsteroidal ligands with high affinity to ERα and ERβ were found. Some compounds were considerably stronger agonists/antagonists at ERα, whereas other compounds were stronger agonists/antagonists at ERβ.

In WO 00/31112, new steroidal compounds based on the building block of the estradiol that is unsubstituted in 8-position are described that carry in 11β-position a hydrocarbon radical that contains an individual linear chain with a length of 5 to 9 carbon atoms. These compounds have an ERα-agonistic/ERβ-antagonistic profile of action. Based on this mixed estrogen receptor profile, these compounds are suitable as improved estrogens for the treatment of estrogen-induced disorders and for contraception together with a gestagen.

In U.S. Ser. No. 60/271409 (un-prepublished), in-vivo findings are shown for the first time from which it is clear that ERβ-selective agonists result in an improvement of the folliculogenesis, whereas ERβ-selective antagonists reduce the fertility, i.e., the ovulation rate.

The object of this invention is therefore to provide compounds that have in vitro a dissociation with respect to the binding to estrogen receptor preparations from rat prostates and rat uteri and that exert a contraceptive action in vivo by their preferential action on the ovary without influencing other estrogen-sensitive organs, such as, e.g., the uterus or the liver. These compounds also are to be used for contraception in men as well as for treating benign or malignant proliferative diseases of the ovary.

This object is achieved by the provision of the compounds of general formula I:

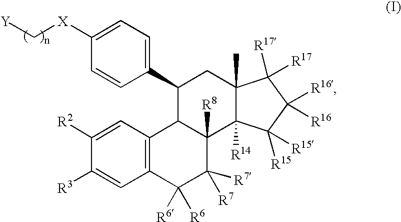

in which

R² means hydrogen, halogen (F, Cl, Br, I);

a radical R¹⁸ or R¹⁸O, whereby R¹⁸ means hydrogen, an alkyl radical or alkanoyl radical (straight-chain or branched-chain, saturated or unsaturated with up to 6 carbon atoms and up to 2 multiple bonds), a benzoyl radical, a trifluoromethyl group;

a radical R¹⁹SO₂O, in which R¹⁹ means an R²⁰R²¹N group, in which R²⁰ and R²¹, independently of one another, mean a hydrogen, a C₁-C₅-alkyl radical, a group C(O)R²², in which R²² means a hydrocarbon radical (optionally substituted, straight-chain or branched-chain, saturated or unsaturated in up to three places, partially or completely halogenated) with up to 10 carbon atoms), an optionally substituted C₃-C₇-cycloalkyl radical, an optionally substituted C₄-C₁₅-cycloalkylalkyl radical or an optionally substituted aryl, heteroaryl or aralkyl radical, or, together with the N-atom, means a polymethylenimino radical with 4 to 6 C atoms or a morpholino radical;

R³ means a radical R¹⁸O, R¹⁹SO₂O or OC(O)R²², with R¹⁸, R¹⁹ and R²² in the meaning that is indicated under R², and in addition R¹⁸ means an aryl, hetaryl or aralkyl radical;

R⁶, R⁷ mean hydrogen;

R⁶ means hydrogen, a hydroxy group, a group R²² in the meaning that is indicated under R²;

R⁷ means hydrogen, halogen, a group R¹⁸O, R¹⁹SO₂O, OC(O)R²², with R¹⁸, R¹⁹, R²² in the meaning that is indicated under R²;

R⁸ means an alkyl radical or alkenyl radical (both straight-chain or branched-chain, optionally partially or completely halogenated, with up to 5 carbon atoms), an ethinyl or prop-1-inyl radical;

X means a direct bond, an oxygen or sulfur atom,

n means an integer from 1 to 12, whereby the corresponding alkylene group can be interrupted by up to 3 O, S or N-methyl,

Y means an amine —NR²⁵R²⁶, with R²⁵ and R²⁶ of the same or a different type, hydrogen, a hydrocarbon radical (optionally substituted, straight-chain or branched-chain, saturated or unsaturated in up to three places, optionally partially or completely halogenated) with up to 10 carbon atoms, an optionally substituted C₃-C₇-cycloalkyl radical, an optionally substituted C₄-C₁₅-cycloalkylalkyl radical or an optionally substituted aryl, heteroaryl or aralkyl radical or together with the nitrogen a heterocyclic compound (optionally with another heteoratom (O, S, N) that is optionally substituted with a C₁-C₄-alkyl radical),

an amide CO—NR²⁵R²⁶ with R²⁵, R²⁶ of the same or a different type, hydrogen, a hydrocarbon radical (optionally substituted, straight-chain or branched-chain, saturated or unsaturated in up to three places, optionally partially or completely halogenated) with up to 10 carbon atoms, an optionally substituted C₃-C₇-cycloalkyl radical, an optionally substituted C₄-C₁₅-cycloalkylalkyl radical or an optionally substituted aryl, heteroaryl or aralkyl radical or together with the nitrogen a heterocyclic compound (optionally with another heteroatom (O, S, N), optionally substituted with a C₁-C₄-alkyl radical),

a grouping —S(O)ₘR²⁷ with m=0, 1 or 2, and R²⁷ as a hydrocarbon radical (optionally substituted, straight-chain or branched-chain, saturated or unsaturated in up to three places, optionally partially or completely halogenated) with up to 10 carbon atoms, an optionally substituted C₃-C₇-cycloalkyl radical, an optionally substituted C₄-C₁₅-cycloalkylalkyl radical or an optionally substituted aryl, heteroaryl or aralkyl radical;

R¹⁴ means hydrogen, an additional bond with R¹⁶;

R¹⁵ means hydrogen, an additional bond with R¹⁴, R¹⁶;

R¹⁶ means hydrogen, a bond with R¹⁵;

R^{15′}, R^{16′}, independently of one another, mean hydrogen, halogen, a group R¹⁸O, R¹⁹SO₂O or OC(O)R²², with R¹⁸, R¹⁹ and R²² in each case in the meaning that is indicated under R²;

R¹⁷, R^{17′} each mean a hydrogen atom;

a hydrogen atom and a halogen atom;

a hydrogen atom and a benzyloxy group;

a hydrogen atom and a group R¹⁹SO₂—O—;

a group R¹⁸ and a group —C(O)R²² or —O—C(O)R²²;

a group R¹⁸—O— and a group —O—C(O)R²², in each case with R¹⁸, R¹⁹ and R²² in the meaning that is indicated under R²;

R¹⁷, R^{17′} together mean a group ═CR²³R²⁴, in which R²³ and R²⁴, independently of one another, represent a hydrogen atom and a halogen atom, or together represent an oxygen atom.

The possible substituents at carbon atoms 6, 7, 15, 16 and 17 can be respectively in α- or β-position.

In the compounds of general formula I as well as in the claimed partial structures, a fluorine, chlorine, bromine or iodine atom can always stand for a halogen atom; a fluorine atom is preferred in each case.

In particular, the hydrocarbon radicals, which can be partially or completely halogenated, are fluorinated radicals.

Hydrocarbon radical R¹⁸ is, for example, a methyl, ethyl, propyl, isopropyl, butyl, isobutyl, tert-butyl, pentyl, isopentyl, neopentyl, heptyl or hexyl radical.

Alkoxy group OR¹⁸ can contain 1 to 6 carbon atoms, whereby methoxy, ethoxy, propoxy, isopropoxy and t-butyloxy groups are preferred.

Representatives of the C₁-C₅-alkyl radicals R²⁰ and R²¹ are methyl, ethyl, propyl, isopropyl, butyl, isobutyl, tert-butyl, pentyl, isopentyl and neopentyl.

As representatives of straight-chain or branched-chain hydrocarbon radicals R²² with 1 to a maximum of 10 carbon atoms, for example, methyl, ethyl, propyl, isopropyl, butyl, isobutyl, tert-butyl, pentyl, isopentyl, neopentyl, heptyl, hexyl and decyl can be mentioned; methyl, ethyl, propyl and isopropyl are preferred.

As perfluorinated alkyl groups, for example, trifluoromethyl, pentafluoroethyl and nonafluorobutyl can be mentioned. Representatives of the partially fluorinated alkyl groups are, for example, 2,2,2-trifluoroethyl, 5,5,5,4,4-pentafluoropentyl, 6,6,6,5,5,4,4,3,3-nonafluorohexyl, etc.

As a C₃-C₇-cycloalkyl group, a cyclopropyl, butyl, pentyl, hexyl or heptyl group can be mentioned.

A C₄-C₁₅-cycloalkylalkyl radical has 3 to 7 carbon atoms in the cycloalkyl portion; typical representatives are the cycloalkyl groups that are mentioned directly above. The alkyl portion has up to 8 carbon atoms.

As examples of a C₄-C₁₅-cycloalkylalkyl radical, the cyclopropylmethyl, cyclopropylethyl, cyclopentylmethyl, cyclopentylpropyl group, etc., can be mentioned.

In terms of this invention, an aryl radical is a phenyl, 1- or 2-naphthyl radical; the phenyl radical is preferred.

Examples of a heteroaryl radical are the 2-, 3- or 4-pyridinyl, the 2- or 3-furyl, the 2- or 3-thienyl, the 2- or 3-pyrrolyl, the 2-, 4- or 5-imidazolyl, the pyrazinyl, the 2-, 4- or 5-pyrimidinyl or 3- or 4-pyridazinyl radical.

As substituents for an aryl or heteroaryl radical, for example, a methyl-, ethyl-, trifluoromethyl-, pentafluoroethyl-, trifluoromethylthio-, methoxy-, ethoxy-, nitro-, cyano-, halogen-(fluorine, chlorine, bromine, iodine), hydroxy-, amino-, mono(C₁₋₈ alkyl) or di(C₁₋₈ alkyl)amino, whereby both alkyl groups are identical or different, di(aralkyl)amino, whereby both aralkyl groups are identical or different, can be mentioned.

An aralkyl radical is a radical that contains in the ring up to 14, preferably 6 to 10, C atoms and in the alkyl chain 1 to 8, preferably 1 to 4, C atoms. Thus, as aralkyl radicals, for example, benzyl, phenylethyl, naphthylmethyl, naphthylethyl, furylmethyl, thienylethyl, and pyridylpropyl are suitable. The rings can be substituted in one or more places by halogen, OH, O-alkyl, CO₂H, CO₂alkyl, —NO₂, —N₃, —CN, C₁-C₂₀ alkyl, C₁-C₂₀ acyl, C₁-C₂₀ acyloxy groups.

The alkyl groups or hydrocarbon radicals can be partially or completely fluorinated or substituted by 1-5 halogen atoms, hydroxy groups or C₁-C₄-alkoxy groups.

A vinyl radical or allyl radical is primarily defined with a C₂-C₅-alkenyl radical; the former is especially preferred.

One or more hydroxyl groups at C atoms 3, 16 and 17 can be esterified with an aliphatic, straight-chain or branched-chain, saturated or unsaturated C₁-C₁₄ mono- or polycarboxylic acid or an aromatic carboxylic acid or with an α- or β-amino acid.

Suitable as such carboxylic acids for esterification are, for example:

Monocarboxylic acids: formic acid, acetic acid, propionic acid, butyric acid, isobutyric acid, valeric acid, isovaleric acid, pivalic acid, lauric acid, myristic acid, acrylic acid, propiolic acid, methacrylic acid, crotonic acid, isocrotonic acid, oleic acid, and elaidic acid.

Esterification with acetic acid, valeric acid or pivalic acid is preferred.

Dicarboxylic acids: oxalic acid, malonic acid, succinic acid, glutaric acid, adipic acid, pimelic acid, suberic acid, azelaic acid, sebacic acid, maleic acid, fumaric acid, muconic acid, citraconic acid, and mesaconic acid.

Aromatic carboxylic acids: benzoic acid, phthalic acid, isophthalic acid, terephthalic acid, naphthoic acid, o-, m- and p-toluic acid, hydratropic acid, atropic acid, cinnamic acid, nicotinic acid, and isonicotinic acid.

Esterification with benzoic acid is preferred.

As amino acids, the representatives of these classes of substances that are known sufficiently to one skilled in the art are suitable, for example, alanine, β-alanine, arginine, cysteine, cystine, glycine, histidine, leucine, isoleucine, phenylalanine, proline, etc.

Esterification with β-alanine is preferred.

According to the invention, those compounds of general formula I are preferred in which

R6, R6′, R7, R7′, R14, R15 and R15′ in each case mean a hydrogen atom, and

X means an oxygen or sulfur atom.

Of these last-mentioned compounds, in turn those compounds are preferred in which X stands for an oxygen atom.

Another variant of the invention are estratriene derivatives of general formula I in which

R¹⁷ and R^{17′} mean a group R¹⁸—O— and a group R¹⁸—; a group R¹⁸- and a group —O—C(O)R²², with R¹⁸ and R²² in each case in the meaning that is indicated under R².

Of these last-mentioned groups, those are preferred in which

R¹⁷ and R^{17′} are a hydroxy group and a hydrogen atom, a C₁-C₄-alkyl group or a C₂-C₄-alkenyl group

and especially preferred are those

in which

R¹⁷ and R^{17′} are a hydroxy group and a hydrogen atom, a methyl, ethinyl or prop-1-inyl group.

According to another variant of the invention, the substituent —X—(—)ₙ—Y on the 11β-phenyl radical is selected from the group of substituents 2-(dimethylamino)-ethoxy, 2-(N-methyl-N-phenyl-amino)ethoxy), 2-(1-piperidinyl)ethoxy, 2-(1-pyrrolidinyl)ethoxy, 2-(1-morpholinyl)ethoxy, N-butyl-N-methyl-8-octanamidoyl, N-isopropyl-N-methyl-8-octanamidoyl, 5-[(4,4,5,5,5-pentafluoropentyl)sulfonyl]pentyloxy.

Preferred according to the invention are the compounds

8β-Methyl-11β-[4-(2-N-piperidinyl-ethoxy)-phenyl]-estra-1,3,5(10)-triene-3,17β-diol

11β-[4-(N,N,-dimethyl-ethoxy-phenyl]-8β-methyl-estra-1,3,5(10)-triene-3,17β-diol

8β-methyl-11β-[4-(2-N-morpholinyl-ethoxy)-phenyl]-estra-1,3,5(10)-triene-3,17β-diol

8β-methyl-11β-[4-[2-(1-pyrrolidinyl)ethoxy]phenyl]-estra-1,3,5(10)-triene-3,17β-diol

8β-methyl-11β-[4-[2-(N-methyl-N-phenyl-amino)ethoxy]-estra-1,3,5(10)-triene-3,17β-diol

8β-methyl-11β-[4-(2-N-piperidinyl-ethoxy)-phenyl]-estra-1,3,5(10)-triene-3,17β-diol-3-sulfamate

11β-[4-(N,N,-dimethyl-ethoxy)-phenyl]-8β-methyl-estra-1,3,5(10)-triene-3,17β-diol-3-sulfamate

8β-methyl-11β-[4-(2-N-morpholinyl-ethoxy)-phenyl]-estra-1,3,5(10)-triene-3,17β-diol-3-sulfamate

8β-methyl-11β-[4-[2-(1-pyrrolidinyl)ethoxy]phenyl]-estra-1,3,5(10)-triene-3,17β-diol-3-sulfamate

8β-methyl-11β-[4-[2-(N-methyl-N-phenyl-amino)ethoxy]-estra-1,3,5(10)-triene-3,17β-diol-3-sulfamate

8β-methyl-11β-[4-[2-(2-N-piperidinyl-ethoxy)-phenyl]-estra-1,3,5(10)-triene-3,17β-diol-3-acetate

11β-[4-(N,N,-dimethyl-ethoxy)-phenyl]-8β-methyl-estra-1,3,5(10)-triene-3,17β-diol-3-acetate

8β-methyl-11β-[4-(2-N-morpholinyl-ethoxy)-phenyl]-estra-1,3,5(10)-triene-3,17β-diol-3-acetate

8β-methyl-11β-[4-[2-(1-pyrrolidinyl)ethoxy]phenyl]-estra-1,3,5(10)-triene-3,17β-diol-3-acetate

8β-methyl-11β-[4-[2-(N-methyl-N-phenyl-amino)ethoxy]-estra-1,3,5(10)-triene-3,17β-diol-3-acetate

11β-[4-(2-N-piperidinyl-ethoxy)-phenyl]-8β-vinyl-estra-1,3,5(10)-triene-3,17β-diol

11β-[4-(N,N,-dimethyl-ethoxy)-phenyl]-8β-vinyl-estra-1,3,5(10)-triene-3,17β-diol

11β-[4-(2-N-morpholinyl-ethoxy)-phenyl]-8β-vinyl-estra-1,3,5(10)-triene-3,17β-diol

11β-[4-[2-(1-pyrrolidinyl)ethoxy]-8β-vinyl-estra-1,3,5(10)-triene-3,17β-diol

11β-[2-(N-methyl-N-phenyl-amino)ethoxy]-8β-vinyl-estra-1,3,5(10)-triene-3,17β-diol

11β-[4-(2-N-piperidinyl-ethoxy)-phenyl]-8β-vinyl-estra-1,3,5(10)-triene-3,17β-diol-3-sulfamate

11β-[4-(N,N,-dimethyl-ethoxy)-phenyl]-8β-vinyl-estra-1,3,5(10)-triene-3,17β-diol-3-sulfamate

11β-[4-(2-N-morpholinyl-ethoxy)-phenyl]-8β-vinyl-estra-1,3,5(10)-triene-3,17β-diol-3-sulfamate

11β-[4-[2-(1-pyrrolidinyl)ethoxy]-8β-vinyl-estra-1,3,5(10)-triene-3,17β-diol-3-sulfamate

11β-[2-(N-methyl-N-phenyl-amino)ethoxy]-8β-vinyl-estra-1,3,5(10)-triene-3,17β-diol-3-sulfamate

11β-[4-(2-N-piperidinyl-ethoxy)-phenyl]-8β-vinyl-estra-1,3,5(10)-triene-3,17β-diol-3-acetate

11β-[4-(N,N,-dimethyl-ethoxy)phenyl]-8β-vinyl-estra-1,3,5(10)-triene-3,17β-diol-3-acetate

11β-[4-(2-N-morpholinyl-ethoxy)-phenyl]-8β-vinyl-estra-1,3,5(10)-triene-3,17β-diol3-acetate

11β-[4-[2-(1-pyrrolidinyl)ethoxy]-8β-vinyl-estra-1,3,5(10)-triene-3,17β-diol-3-acetate

11β-[2-(N-methyl-N-phenyl-amino)ethoxy]-8β-vinyl-estra-1,3,5(10)-triene-3,17β-diol-3-acetate

11β-[4-[7-(N-isopropyl-N-methylamido)-heptyloxy]-8β-methyl-estra-1,3,5(10)-triene-3,17β-diol

11β-[4-[7-(N-butyl-N-methylamido)-heptyloxy]-phenyl]-8β-methyl-estra-1,3,5(10)-triene-3,17β-diol

11β-[4-[7-(N-isopropyl-N-methylamido)-heptyloxy]-phenyl]-8β-vinyl-estra-1,3,5(10)-triene3,17β-diol

11β-[4-[7-(N-butyl-N-methylamido)-heptyloxy]-phenyl]-8β-vinyl-estra-1,3,5(10)-triene-3,17β-diol

11β-[4-[7-(N-isopropyl-N-methylamido)-heptyloxy]-phenyl]-8β-methyl-estra-1,3,5(10)-triene-3,17β-diol-3-sulfamate

11β-[4-[7-(N-butyl-N-methylamido-heptyloxy]-phenyl-8β-methyl-estra-1,3,5(10)-triene-3,17β-diol-3-sulfamate

11β-[4-[7-(N-isopropyl-N-methylamido)heptyloxy]-phenyl]-8β-vinyl-estra-1,3,5(10)triene-3,17β-diol-3-sulfamate

11β-[4-[7-(N-butyl-N-methylamido)-heptyloxy]-phenyl]-8β-vinyl-estra-1,3,5(10)-triene-3,17β-diol-3-sulfamate

11β-[4-[7-(N-isopropyl-N-methylamido)-heptyloxy]-phenyl]-8β-methyl-estra-1,3,5(10)-triene-3,17β-diol-3-acetate

11β-[4-[7-(N-butyl-N-methylamido)-heptyloxy]-phenyl]-8β-methyl-estra-1,3,5(10)-triene-3,17β-diol-3-acetate

11β-[4-[7-(N-isopropyl-N-methylamido)-heptyloxy]-phenyl]-8β-vinyl-estra-1,3,5(10)-triene-3,17β-diol-3-acetate

11β-[4-[7-(N-butyl-N-methylamido)-heptyloxy]-phenyl]-8β-vinyl-estra-1,3,5(10)-triene-3,17β-diol-3-acetate

8β-methyl-11β-[4-[5-[(4,4,5,5,5-pentafluoropentyl)-thio]-pentyloxy]-phenyl]-estra-1,3,5(10)-triene-3,17β-diol

11β-[4-[5-[(4,4,5,5,5-pentafluoropentyl)-thio]-pentyloxy]-phenyl]-8β-vinyl-estra-1,3,5(10)-triene-3,17β-diol

8β-methyl-11β-[4-[5-[(4,4,5,5,5-pentafluoropentyl)-sulfinyl]-pentyloxy]-phenyl]-estra-1,3,5(10)-triene-3,17β-diol

11β-[4-[5-[(4,4,5,5,5-pentafluoropentyl)-sulfinyl]-pentyloxy]-phenyl]-8β-vinyl-estra-1,3,5(10)-triene-3,17β-diol

8β-methyl-11β-[4-[5-[(4,4,5,5,5-pentafluoropentyl)-sulfonyl]-pentyloxy]-phenyl]-estra-1,3,5(10)-triene-3,17β-diol

11β-[4-[5-[(4,4,5,5,5-pentafluoropentyl)-sulfonyl]-pentyloxy]-phenyl]-8β-vinyl-estra-1,3,5(10)-triene-3,17β-diol

For the formation of pharmaceutically compatible salts of the compounds of general formula I according to the invention, as inorganic acids, i.a., hydrochloric acid, hydrobromic acid, sulfuric acid and phosphoric acid are considered, and as organic acids, i.a., acetic acid, propionic acid, maleic acid, fumaric acid, succinic acid, benzoic acid, ascorbic acid, oxalic acid, salicylic acid, tartaric acid, citric acid, lactic acid, malic acid, mandelic acid, cinnamic acid and methanesulfonic acid are considered.

The new compounds are suitable for inhibiting folliculogenesis and ovulation, for male contraception and for treating benign and malignant proliferative diseases of the ovary.

Unlike in the estrogen ethinylestradiol that is commonly used for hormonal contraception or else in the compounds that are to be used for contraception according to WO 00/31112, the compounds of general formula I according to the invention can be used by themselves, i.e., without the additional administration of gestagens for contraception.

As prodrugs, the esters of the 8β-substituted estratrienes according to the invention may have advantages compared to the unesterified active ingredients with respect to their method of administration, their type of action, strength and duration of action.

The sulfamates of 8β-substituted estratrienes according to the invention also have pharmacokinetic and pharmacodynamic advantages. Related effects were already described in other steroid-sulfamates (J. Steroid Biochem. Molec. Biol, 55, 395-403 (1995); Exp. Opinion Invest. Drugs 7, 575-589 (1998)).

In this patent application, steroids on which the 8β-substituted estra-1,3,5(10)-triene skeleton is based and which are substituted in 11-position with a β-position p-substituted aryl radical are described for contraception, which have in-vitro dissociation with respect to binding to estrogen receptor preparations from rat prostates and rat uteri and which have in vivo preferably an inhibition of folliculogenesis and ovulation: these substances have a contraceptive action over a wide dose range without influencing other estrogen-sensitive organs, such as, e.g., the uterus or the liver. Moreover, these compounds can be used for male contraception and for treatment of benign or malignant proliferative diseases of the ovary.

The invention also relates to pharmaceutical preparations that contain at least one compound of general formula I (or physiologically compatible addition salts with organic and inorganic acids thereof) for the production of pharmaceutical agents, especially for the indications below.

The compounds can be used for the following indications both after oral and parenteral administration.

The novel selective estrogens that are described in this patent can be used as individual components in pharmaceutical preparations or in combination especially with GnRH-antagonists, progesterone receptor antagonists, mesoprogestins or gestagens or tissue-selective gestagens (action on A/B-form type).

The substances and the pharmaceutical agents that contain them are especially suitable for ovarian contraception, for the treatment of benign or malignant proliferative diseases of the ovary, such as, e.g., ovarian cancer, and granulosa cell tumors.

In addition, the compounds can be used for treating male fertility disorders and prostatic diseases.

The amount of a compound of general formula I′ that is to be administered varies within a wide range and can cover any effective amount. On the basis of the condition that is to be treated and the type of administration, the amount of the compound that is administered can be 0.01 μg/kg-10 mg/kg of body weight, preferably 0.04 μg/kg-1 mg/kg of body weight, per day.

In humans, this corresponds to a dose of 0.8 μg to 8 g, preferably 3.2 μg to 80 mg, daily.

According to the invention, a dosage unit contains 1.6 μg to 2000 mg of one or more compounds of general formula I′.

The compounds according to the invention and the acid addition salts are suitable for the production of pharmaceutical compositions and preparations. The pharmaceutical compositions or pharmaceutical agents contain as active ingredients one or more of the compounds according to the invention or their acid addition salts, optionally mixed with other pharmacologically or pharmaceutically active substances. The production of the pharmaceutical agents is carried out in a known way, whereby the known and commonly used pharmaceutical adjuvants as well as other commonly used vehicles and diluents can be used.

As such vehicles and adjuvants, for example, those are suitable that are recommended or indicated in the following bibliographic references as adjuvants for pharmaceutics, cosmetics and related fields: Ullmans Encyklopädie der technischen Chemie [Ullman's Encyclopedia of Technical Chemistry], Volume 4 (1953), pages 1 to 39; Journal of Pharmaceutical Sciences, Volume 52 (1963), page 918 ff., issued by Czetsch-Lindenwald, Hilfsstoffe für Pharmazie und angrenzende Gebiete [Adjuvants for Pharmaceutics and Related Fields]; Pharm. Ind., Issue 2, 1961, p. 72 and ff.: Dr. H. P. Fiedler, Lexikon der Hilfsstoffe für Pharmazie, Kosmetik und angrenzende Gebiete [Dictionary of Adjuvants for Pharmaceutics, Cosmetics and Related Fields], Cantor K G, Aulendorf in Württemberg 1971.

The compounds can be administered orally or parenterally, for example intraperitoneally, intramuscularly, subcutaneously or percutaneously. The compounds can also be implanted in the tissue.

For oral administration, capsules, pills, tablets, coated tablets, etc., are suitable. In addition to the active ingredient, the dosage units can contain a pharmaceutically compatible vehicle, such as, for example, starch, sugar, sorbitol, gelatin, lubricant, silicic acid, talc, etc.

For parenteral administration, the active ingredients can be dissolved or suspended in a physiologically compatible diluent. As diluents, very often oils with or without the addition of a solubilizer, a surfactant, a suspending agent or an emulsifying agent are used. Examples of oils that are used are olive oil, peanut oil, cottonseed oil, soybean oil, castor oil and sesame oil.

The compounds can also be used in the form of a depot injection or an implant preparation, which can be formulated so that a delayed release of active ingredient is made possible.

As inert materials, implants can contain, for example, biodegradable polymers, or synthetic silicones such as, for example, silicone rubber. In addition, for percutaneous administration, the active ingredients can be added to, for example, a patch.

For the production of intravaginal systems (e.g., vaginal rings) or intrauterine systems (e.g., pessaries, coils, IUDs, Mirena^{(R)}) that are loaded with active compounds of general formula I for local administration, various polymers are suitable, such as, for example, silicone polymers, ethylene vinyl acetate, polyethylene or polypropylene.

To achieve better bio-availability of the active ingredient, the compounds can also be formulated as cyclodextrin clathrates. For this purpose, the compounds are reacted with α-, β-, or γ-cyclodextrin or derivatives of the latter (PCT/EP95/02656).

According to the invention, the compounds of general formula I can also be encapsulated with liposomes.

Methods

Estrogen Receptor Binding Studies

The binding affinity of the new selective estrogens was tested in competitive experiments with use of 3H-estradiol as a ligand to estrogen receptor preparations from rat prostates and rat uteri. The preparation of prostate cytosol and the estrogen receptor test with prostate cytosol was carried out as described by Testas et al. (1981) (Testas, J. et al., 1981, Endocrinology 109: 1287-1289).

The preparation of rat uterus cytosol as well as the receptor test with the ER-containing cytosol were basically performed as described by Stack and Gorski, 1985 (Stack, Gorski 1985, Endocrinology 117, 2024-2032) with some modifications as described in Fuhrmann et al. (1995) (Fuhrmann, U. et al. 1995, Contraception 51: 45-52).

The substances that are described in this industrial property have higher binding affinity to the estrogen receptor from rat prostates than to estrogen receptors from rat uteri. In this case, it is assumed that ERβ predominates in the rat prostates over ERα, and ERα predominates in rat uteri over ERβ. Table 1 shows that the ratio of the binding to prostate and uterus receptors qualitatively coincides with the quotient of relative binding affinity (RBA) to human ERβ and ERα of rats (according to Kuiper et al. (1996), Endocrinology 138: 863-870) (Table 1).

TABLE 1

| | | | | | Rat | Rat | prost. |
|---|---|---|---|---|---|---|---|
| | | hERα | hERβ | ERβ/ | uterus | prost. | ER/uterus |
| Estrogen | Structure | RBA* | RBA | ERα | ER(RBA) | ER(RBA) | ER |
| Estradiol | 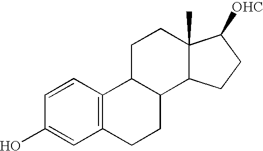 | 100 | 100 | 1 | 100 | 100 | 1 |
| Estrone | 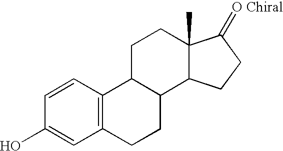 | 60 | 37 | 0.6 | 3 | 2 | 0.8 |
| 17α-Estradiol | 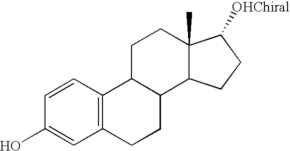 | 58 | 11 | 0.2 | 2.4 | 1.3 | 0.5 |
| Estriol | 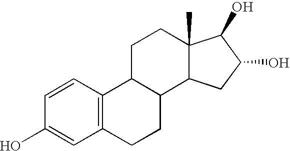 | 14 | 21 | 1.5 | 4 | 20 | 5 |
| 5-Androstene-diol | 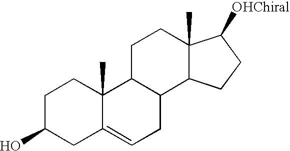 | 6 | 17 | 3 | 0.1 | 5 | 50 |
| Genisteine | 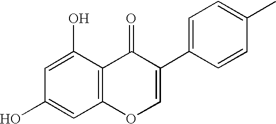 | 5 | 36 | 7 | 0.1 | 10 | 100 |
| Coumestrol | 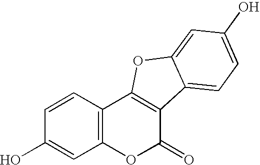 | 94 | 185 | 2 | 1.3 | 24 | 18 |

*Cited from: Kuiper et al. (1996), Endocrinology 138: 863-870

Sample Studies of Contraceptive Action

Study of Early Folliculogenesis:

Immature female rats are hypophysectomized. This day is defined as day 0. From day 1-day 4, subcutaneous and/or oral treatment is carried out with the active substance in combination with 17β-estradiol. The animals were autopsied on day 5. The ovary is removed and analyzed macroscopically, e.g., organ weights, and microscopically, e.g., histological evaluation of the follicles, so-called follicle staging.

Study of Late Folliculogenesis/Ovulation

Immature female rats are hypophysectomized. This day is defined as day 0. From day 1-day 4, subcutaneous and/or oral treatment is carried out with the active substance in combination with 17β-estradiol. On day 5, a subcutaneous injection with PMSG (pregnant mare serum gonadotrophin) is carried out. On day 7, hCG is administered intraperitoneally to trigger ovulation. On day 8, the ovary is removed and analyzed macroscopically (e.g., ovary weights) and/or microscopically (e.g., histological evaluation of the follicles, so-called follicle staging). The tubes are flushed and checked for the presence of egg cells.

Study of Ovulation

Immature female rats are treated (day 1) subcutaneously with PMSG (pregnant mare serum gonadotrophin) at the age of 23 days. On the same day, as well as 24 and 48 hours later, the animals receive the active substance, administered subcutaneously or orally. 54 hours after the PMSG injection, the animals receive an intraperitoneal injection of hCG to trigger ovulation. Autopsy is carried out 16 hours after the hCG is administered. The tubes are flushed and checked for the presence of egg cells.

Another possibility to detect in vivo the dissociated estrogenic action of the substances according to the invention consists in the fact that after a one-time administration of the substances in rats, effects on the expression of 5HT2a-receptor and serotonin transporter protein and mRNA levels in ERβ-rich brain areas can be measured. Compared to the effect on the serotonin receptor and transporter expression, the effect on the LH-secretion is measured. Substances with higher binding to the rat prostate—compared to the rat uterus estrogen receptor—are more potent with respect to increasing the expression of serotonin receptors and transporters, in comparison to their positive effect on the LH release. The density of serotonin receptors and transporters is determined in brain sections using radioactive ligands, and the corresponding mRNA is determined using in-situ hybridization. The method is described in the literature: G. Fink & B. E. H. Sumner 1996 Nature 383: 306; B. E. H. Sumner et al. 1999 Molecular Brain Research, in press.

Production of the Compounds According to the Invention

The compounds of general formula I according to the invention are produced as described in the examples. Additional compounds of general formula I can be obtained by an analogous procedure using reagents that are homologous to the reagents that are described in the examples.

Etherification and/or esterification of free hydroxy groups is carried out according to methods that are common to one skilled in the art.

The compounds according to the invention can be present in carbon atoms 6, 7, 15, 16 and 17 as α,β-stereoisomers. In the production of compounds according to the described processes, the compounds in most cases accumulate as mixtures of the corresponding α,βisomers. The mixtures can be separated by, for example, chromatographic processes.

According to general formula I, possible substituents can already be present in final form or in the form of a precursor even in the starting product, a substituted estrone already corresponding to the desired end product.

The introduction of a substituent or reactive precursor on carbon atom 7 by nucleophilic addition of the substituent or precursor on a 6-vinylsulfone thus is possible (DE 42 18 743 A1). In this case, 7α- and 7β-substituted compounds, which can be separated by, for example, chromatographic processes, are obtained in different proportions, based on the reactants and the selected reaction conditions.

17-Substituents are also introduced according to known processes by nucleophilic addition of the desired substituent or a reactive precursor thereof and optionally further built up.

The 8β-substituted estratriene-carboxylic acid esters according to the invention are produced from the corresponding hydroxy steroids analogously to processes that are also known (see, e.g., Pharmazeutische Wirkstoffe, Synthesen, Patente, Anwendungen [Pharmaceutical Active Ingredients, Syntheses, Patents, Applications]; A. Kleemann, J. Engel', Georg Thieme Verlag Stuttgart 1978. Arzneimittel, Fortschritte [Pharmaceutical Agents, Advances] 1972 to 1985; A. Kleemann, E. Lindner, J. Engel (Editors), VCH 1987, pp. 773-814).

The estratriene-sulfamates according to the invention are available in a way that is known in the art from the corresponding hydroxy steroids by esterification with sulfamoyl chlorides in the presence of a base (Z. Chem. 15, 270-272 (1975); Steroids 61, 710-717 (1996)).

Subsequent acylation of the sulfamide group results in the (N-acyl)sulfamates according to the invention, for which pharmacokinetic advantages were already detected in the case of the absence of an 8-substituent (cf. DE 195 40 233 A1).

The regioselective esterification of polyhydroxylated steroids with N-substituted and N-unsubstituted sulfamoyl chlorides is carried out according to partial protection of those hydroxyl groups that are to remain unesterified. Silyl ethers have turned out to be protective groups with selective reactivity that is suitable for this purpose, since these silyl ethers are stable under the conditions of sulfamate formation, and the sulfamate group remains intact when the silyl ethers are again cleaved off for regeneration of the residual hydroxyl group(s) still contained in the molecule (Steroids 61, 710-717 (1996)).

The production of the sulfamates according to the invention with one or more additional hydroxyl groups in the molecule is also possible in that the starting material is suitable hydroxy-steroid ketones. First, depending on the goal, one or more hydroxyl groups that are present are subjected to sulfamoylation. Then, the sulfamate groups optionally can be converted with a desired acyl chloride in the presence of a base into the (N-acyl)sulfamates in question. The now present oxosulfamates or oxo-(N-acyl)sulfamates are converted by reduction into the corresponding hydroxysulfamates or hydroxy-(N-acyl)sulfamates (Steroids 61, 710-717 (1996)). Sodium borohydride and the borane-dimethyl sulfide complex are suitable as proper reducing agents.

Functionalizations at carbon atom 2 are possible by, for example, electrophilic substitution after prior deprotonation of the 2-position of the corresponding 3-(2-tetrahydropyranyl)- or 3-methyl ether with a lithium base (e.g., methyllithium, butyllithium). Thus, for example, a fluorine atom can be introduced by reaction of the C—H-activated substrate with a fluorinating reagent such as N-fluoromethane sulfonimide (WO 94/24098).

The introduction of variable substituents in rings B and D of the estratriene skeleton can basically be carried out according to the chemical teaching that is known to one skilled in the art, with which the corresponding estratriene derivatives that are not substituted in 8-position are produced (see, i.a.: Steroide [Steroids], L. F. Fieser, M. Fieser, Verlag Chemie, Weinheim/Bergstr., 1961; Organic Reactions in Steroid Chemistry, J. Fried, J. A. Edwards, Van Nostrand Reinhold Company, New York, Cincinnati, Toronto, London, Melbourne, 1972; Medicinal Chemistry of Steroids, F. J. Zeelen, Elsevier, Amsterdam, Oxford, New York, Tokyo, 1990). This relates to, for example, the introduction of substituents, such as hydroxyl or alkyloxy groups, alkyl, alkenyl or alkinyl groups or halogen, especially fluorine.

Substituents according to general formula I can also be introduced in the stage of estratrienes that are already substituted in 8-position, however. This can be useful or necessary especially in the case of multiple substitutions of the desired final compound.

The examples below are used for a more detailed explanation of the invention.

As starting material for such syntheses, 11-keto-estratetraene derivatives (U.S. Pat. No. 3,491,089, Tetrahedron Letters, 1967, 37, 3603), which are substituted stereoselectively in 8β-position in the reaction with diethylaluminum cyanide, are used. By conversion into a Δ-9,11-enol triflate and subsequent Stille coupling, 8β-substituted 11-aryl-estra-1,3,5(10),9(11)-tetraenes are obtained. The 8β-cyano grouping can then be converted into the 8β-aldehyde. After hydrogenation of the C(9)-C(11) double bond has taken place, a functionalization (e.g., by Wittig reactions) results in the 8β-steroids according to the invention.

The 8β-substituted 11-aryl-estra-1,3,5(10),9(11)-tetraenes that are first obtained in this sequence can be further reacted to form many substitution patterns on the steroid like the 8β-substituted 11β-aryl-estra-1,3,5(10)-trienes according to the methods that are known to one skilled in the art.

For the production of the derivatives of 8β-substituted estra-1,3,5(10)-triene-3,16ξ-diols according to the invention without 17-substituents, mainly the following synthesis strategy is used. In this connection, the 8β-carbonyl function is protected as an acetal. After subsequent oxidation, the 17-keto steroid can be converted into a sulfonylhydrazone, in the simplest case by reaction with phenylsulfonyl hydrazide. By a degradation reaction, the formation of the C(16)-C(17) olefin is carried out (Z. Chem. 1970, 10, 221-2; Liebigs Ann. Chem. 1981, 1973-81), in which hypobromide is stored in a regio/stereocontrolled way. Reductive dehalogenation and removal of the acetal protective group at 8β opens the way for transformations to the compounds according to the invention. The 16β-alcohols that can be obtained according to this method can be converted into the 16α-epimer by known methods (Synthesis 1980, 1).

Another variant for the introduction of the hydroxyl group at C-atom 16 consists in the hydroboration of the 16(17)-double bond with sterically exacting boranes. Of this reaction, it is known that it results in 16-oxidized products (Indian J. Chem. 1971, 9, 287-8). The reaction of estra-1,3,5(10),16-tetraenes with 9-borabicyclo[3.3.1 ]nonane after the oxidation with alkaline hydrogen peroxide consequently produces 16α-hydroxyestratrienes. The epimeric 16β-hydroxy steroids are formed to a lesser extent in this reaction. Further transformations on 8β substituents then result in the compounds of general formula I according to the invention.

Characteristic, but not limiting synthesis processes, which are useful for providing representative substitution patterns on the estrone skeleton, also in combination with several substituents, are found in, for example: C(1) J. Chem. Soc. (C) 1968, 2915; C(7) Steroids 54, 1989, 71; C(8β) Tetrahedron Letters 1964, 1763; J. Org. Chem. 1970, 35, 468; C(15) J. Chem. Soc. Perk. 1 1996, 1269.); C(14β) Z. Chem. 23, 1983, 410.

In the examples and in the diagrams, the following abbreviations apply:

THP=tetrahydropyran-2-yl; Me=methyl; iPr=isopropyl; nBu=nbutyl; Ac=acetyl; Bn=benzyl; Pd₂dba₃=tris(dibenzylideneacetone)-dipalladium

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLE 1

3-Methoxy-8β-methyl-17β-(tetrahydropyran-2-yloxy)-11-trifluoromethanesulfonyloxy-estra-1,3,5(10),9,(11)-tetraene (2)

6 ml of trifluoromethanesulfonic acid anhydride is added in drops [3] at room temperature to 9.2 g of 2,6-di-tert-butyl-4-methylpyridine in 44 ml of pyridine. After the heat shading has run its course, a solution of 3.9 g of ketone 1 [1] in 20 ml of pyridine is added in drops to this solution, and it is stirred for another 2 hours. Pyridine is distilled off with toluene as a co-solvent, the residue is suspended in diethyl ether and filtered. The filtrate is washed with 2N hydrochloric acid, water, saturated sodium bicarbonate solution and saturated sodium chloride solution, dried on magnesium sulfate and concentrated by evaporation. The residue is purified by column chromatography (cyclohexane/ethyl acetate 5:1), and 2.68 g of triflate 2 is obtained as a colorless foam.

11-14-(Benzyloxy)phenyl]-3-methoxy-8β-methyl-17β-(tetrahydropyran-2-yloxy)-estra-1,3,5(10),9,(11)-tetraene (3)

640 mg of lithium chloride and 262 mg of catalyst Pd₂dba₃ are added in succession to 2.68 g of triflate 2 in 26 ml of N-methylpyrrolidinone, and the mixture is stirred for 10 minutes at room temperature [4]. Then, a solution of 2.86 g of aryltributyltin (40) in 24 ml of N-methylpyrrolidinone is added in drops, the reaction mixture is heated to 60° C. and stirred until conversion is completed. It is diluted with ethyl acetate, washed with water, and the organic phase is stirred for 30 minutes with saturated potassium fluoride solution. After the phase separation is completed, it is dried with magnesium sulfate, and the solvent is distilled off. The purification is carried out by column chromatography (cyclohexane/ethyl acetate 10:1), and 2.14 g of a colorless, viscous oil 3 is obtained.

11β-(4-Hydroxyphenyl)-3-methoxy-8β-methyl-17β-(tetrahydropyran-2-yloxy)-estra-1,3,5(10)-triene (4)

A solution of 2.12 g of 3 in 38 ml of tetrahydrofuran/methanol (1:1) is mixed with 1.88 g of palladium (10% on magnesium carbonate) and stirred under a hydrogen atmosphere (100 bar) until conversion is completed. For working-up, it is filtered on Celite and concentrated by evaporation in a vacuum. The purification is carried out by column chromatography (cyclohexane/ethyl acetate 10:1), and 1.7 g of a colorless foam 4 is obtained.

General Instructions for Introducing an Ethanolamine in the 11β-(4-hydroxyphenyl) Group Under Mitsunobu Conditions [7]

Triphenylphosphine (4 equivalents) and the corresponding ethanolamine (5 equivalents) are added in succession to a solution of the corresponding steroid in toluene (40 ml/mmol) at room temperature. Then, diisopropylazodicarboxylate (4 equivalents) is added in drops, and the reaction solution is heated to 50° C. until conversion is completed. For working-up, the batch is diluted with diethyl ether, washed with water, dried on magnesium sulfate, and the solvent is distilled off. The purification is carried out by column chromatography.,

3-Methoxy-8β-methyl-11β-[4-(2-N-piperidinyl-ethoxy)-phenyl]-17β-(tetrahydropyran-2-yloxy)-estra-1,3,5(10)-triene (11)

200 mg of steroid 4 in the reaction with N-(2-hydroxyethyl)-piperidine analogously to instructions 1.4 yields 176 mg of 11 as a colorless foam.

General Operating Instructions for Ether Cleavage by Means of Boron Trichloride/Tetrabutylammonium Iodide [11]

A corresponding amount of boron trichloride (1.5 equivalents each to decomposing ether, one additional equivalent for each basic grouping) is added in drops to a solution, cooled to −78° C., that consists of the corresponding steroid and tetrabutylammonium iodide (1 equivalent) in dichloromethane (5 ml/mmol). The reaction solution is allowed to heat to 0° C. and stirred until conversion is completed. For working-up, the batch is mixed with ice water and stirred for about 30 minutes, then mixed with saturated sodium bicarbonate solution and extracted several times with dichloromethane. The combined organic phases are dried with magnesium sulfate, and the solvent is distilled off. The residue that is obtained is purified by column chromatography.

8β-Methyl-11β-[4-(2-N-piperidinyl-ethoxy)-phenyl]-estra-1,3,5(10)-triene-3,17β-diol (12)

150 mg of steroid 11 is reacted analogously to instructions 1.5. 79 mg of 12 is obtained as a colorless solid (flash point: 123-125° C.).

### EXAMPLE 2

11β-[4-(N,N,-Dimethyl-ethoxy)-phenyl]-3-methoxy-8β-methyl-17β-(tetrahydropyran-2-yloxy)-estra-1,3,5(10)-triene (13)

200 mg of steroid 4 in the reaction with 2-diethylaminoethanol analogously to instructions 1.4 yields 168 mg of 13 as a colorless foam.

11β-[4-(N,N,-Dimethyl-ethoxy)-phenyl]-8β-methyl-estra-1,3,5(10)-triene-1,17β-diol (14)

150 mg of steroid 13 is reacted analogously to instructions 1.5. 82 mg of 14 is obtained as a colorless solid (flash point: 125-126° C.).

### EXAMPLE 3

3-Methoxy-8β-methyl-11β-[4-(2-N-morpholinyl-ethoxy)-phenyl]-17β-(tetrahydropyran-2-yloxy)-estra-1,3,5(10)-triene (15)

200 mg of steroid 4 in the reaction with 4-(2-hydroxyethyl)-morpholine analogously to instructions 1.4 yields 173 mg of 15 as a colorless foam.

8β-Methyl-11β-[4-(2-N-morpholinyl-ethoxy)-pheny]-estra-1,3,5(10)-triene-3,17β-diol (16)

150 mg of steroid 15 is reacted analogously to instructions 1.5. 76 mg of 16 is obtained as a colorless solid (flash point: 122-123° C.).

### EXAMPLE 4

8β-Cyano-3-methoxy-17β-(tetrahydropyran-2-yloxy)-11-trifluoromethanesulfonyloxy-estra-1,3,5(10),9,(11)-tetraene (6)

6 g of ketone 5 [2] is reacted analogously to instructions 1.1, and after purification by column chromatography (cyclohexane/ethyl acetate 5:1), 4.1 g of triflate 6 is obtained as a colorless foam.

11-[4-(Benzyloxy)phenyl]-8β-cyano-3-methoxy-17β-(tetrahydropyran-2-yloxy)-estra-1,3,5(10),9,(11)-tetraene (7)

4.08 g of triflate 6 is reacted analogously to instructions 1.2, and after purification by column chromatography (cyclohexane/ethyl acetate 10:1), 3.56 g of steroid 7 is obtained as a colorless, viscous oil.

11-[4-(Benzyloxy)phenyl]-8β-formyl-3-methoxy-17β-(tetrahydropyran-2-yloxy)-estra-1,3,5(10),9,(11)-tetraene (8)

6.2 ml of a diisobutylaluminum hydride solution (2 M in toluene) is added in drops [6] to a solution of 3.54 g of nitrile 7 in 60 ml of toluene at −10° C. It is stirred until conversion is completed at 0° C. The reaction solution is mixed in succession with 120 ml of toluene, 12 ml of saturated sodium bicarbonate solution and 1.6 ml of 2-propanol and stirred for several hours at room temperature. It is filtered on Celite, and the filtrate is concentrated by evaporation. 60 ml of ethanol/water (5:1) is dissolved [in] the thus obtained colorless foam, 5.82 g of p-toluene sulfonic acid is added, the reaction solution is heated to 60° C. and stirred until conversion is completed. Then, it is diluted with ethyl acetate, washed with water, saturated sodium bicarbonate solution and saturated sodium chloride solution, dried on magnesium sulfate and concentrated by evaporation. The thus obtained yellow viscous oil is dissolved in 30 ml of dichloromethane without further purification and mixed in succession with 5.6 ml of 3,4-dihydro-2H-pyran and 154 mg of pyridinium-4-toluenesulfonate. Then, the reaction solution is refluxed until conversion is completed. For working-up, it is diluted with dichloromethane, washed with water, saturated sodium bicarbonate solution and saturated sodium chloride solution, dried on magnesium sulfate and concentrated by evaporation. The purification of the thus obtained yellow oil is carried out by column chromatography (cyclohexane/ethyl acetate 10:1). 1.77 g of 8 is obtained as a colorless foam.

8β-Formyl-11β-(4-hydroxyphenyl)-3-methoxy-17β-(tetrahydropyran-2-yloxy)-estra-1,3,5(10)-triene (9)

1.77 g of steroid 8 is reacted analogously to instructions 1.3, and a colorless foam 9 (1.46 g), which is used without further purification in the next stage, is obtained.

11β-(4-Hydroxyphenyl)-3-methoxy-17β-(tetrahydropyran-2-yloxy)-8β-vinyl-estra-1,3,5(10)-triene (10)

1.27 g of sodium hydride (80%) in 22 ml of absolute dimethyl sulfoxide was heated for 1 hour to 70° C. The gray-black solution that was obtained was added in drops at room temperature to a solution of 15.94 g of methyltriphenylphosphonium bromide in 90 ml of absolute dimethyl sulfoxide. The solution was colored yellow-green and was stirred for another hour at room temperature [6]. A solution of 1.46 g of aldehyde 9 in 15 ml of dimethyl sulfoxide was added in drops at room temperature to the solution of ylide. The reaction solution was stirred for 2 hours at 40° C., mixed with water at 0° C. and extracted several times with diethyl ether. The combined organic phases were washed with water and saturated sodium chloride solution, dried on magnesium sulfate and concentrated by evaporation. Purification by column chromatography (cyclohexane/ethyl acetate 5:1) yielded 1.02 g of 10 as a colorless foam.

3-Methoxy-11β-[4-(2-N-piperidinyl-ethoxy)-phenyl]-17β-(tetrahdyropyran-2-yloxy-8β-vinyl-estra-1,3,5(10)-triene (17)

150 mg of steroid 10 in the reaction with N-(2-hydroxyethyl)-piperidine analogously to instructions 1.4 yields 125 mg of 17 as a colorless foam.

11β-[4-(2-N-Piperidinyl-ethoxy)-phenyl]-8β-vinyl-estra-1,3,5(10)-triene-3,17β-diol (18)

120 mg of steroid 17 is reacted analogously to instructions 1.5. 62 mg of 18 is obtained as a colorless solid (flash point: 120-123° C.).

### EXAMPLE 5

11β-[4-(N,N,-Dimethyl-ethoxy)-phenyl]-3-methoxy-17β-(tetrahydropyran-2-yloxy)-8β-vinyl-estra-1,3,5(10)-triene (19)

150 mg of steroid 10 in the reaction with 2-dimethylaminoethanol analogously to instructions 1.4 yields 125 mg of 19 as a colorless foam.

11β-[4-(N,N,-Dimethyl-ethoxy)-phenyl]-8β-vinyl-estra-1,3,5(10)-triene-1,17β-diol (20)

120 mg of steroid 19 is reacted analogously to instructions 1.5. 64 mg of 20 is obtained as a colorless solid (flash point: 123-124° C.).

### EXAMPLE 6

3-Methoxy-11μ-[4-(2-N-morpholinyl-ethoxy)-phenyl]-17β-(tetrahydropyran-2-yloxy)-8β-vinyl-estra-1,3,5(10)-triene (21)

150 mg of steroid 10 in the reaction with 4-(2-hydroxyethyl)-morpholine analogously to instructions 1.4 yields 128 mg of 21 as a colorless foam.

11β-[4-(2-N-Morpholinyl-ethoxy)-phenyl]-8β-vinyl-estra-1,3,5(10)-triene-3,17β-diol (22)

120 mg of steroid 21 is reacted analogously to instructions 1.5. 60 mg of 22 is obtained as a colorless solid (flash point: 119-120° C.).

### EXAMPLE 7

General Operating Instructions for Introducing a Side Chain that is Bromated in ω-Position to the 11β-(4-Hydroxyphenyl) Group under Basic Conditions [8]

1.2 equivalents of a 2N sodium hydroxide solution is added to a solution of corresponding steroid (4, 10) in acetone (5 ml/mmol). The solution is refluxed, and 2.4 equivalents of the corresponding co-bromine side chain [9] is added in portions. The reaction solution is refluxed until conversion is completed and diluted with saturated ammonium chloride solution. Then, it is extracted several times with ethyl acetate, the organic phase is washed neutral with water, dried on magnesium sulfate and concentrated by evaporation. The purification is carried out by column chromatography (cyclohexane/ethyl acetate).

11β-[4-[7-(N-Isopropyl-N-methylamido)-heptyloxy]-phenyl]-3-methoxy-8β-methyl-17β-(tetrahydropyran-2-yloxy)-estra-1,3,5(10)-triene (23)

200 mg of steroid 4 in the reaction with 8-bromo-N-isopropyl-N-methyl-octanamide analogously to instructions 7.1 yields 184 mg of 23 as a colorless foam.

11β-[4-[7-(N-Isopropyl-N-methylamido)-heptyloxy]-phenyl]-8β-methyl-estra-1,3,5(10)-triene-3,17β-diol (24)

160 mg of steroid 23 is reacted analogously to instructions 1.5. 79 mg of 24 is obtained as a colorless, amorphous solid.

### EXAMPLE 8

11β-[4-[7-(N-Butyl-N-methylamido)-heptyloxy]-phenyl]-3-methoxy-8β-methyl-17β-(tetrahydropyran-2-yloxy)-estra-1,3,5(10)-triene (25)

200 mg of steroid 4 in the reaction with 8-bromo-N-butyl-N-methyl-octanamide analogously to instructions 7.1 yields 182 mg of 25 as a colorless foam.

11β-[4-[7-(N-Butyl-N-methylamido)-heptyloxy]-phenyl]-8β-methyl-estra-1,3,5(10)-triene-3,171-diol (26)

160 mg of steroid 25 is reacted analogously to instructions 1.5. 81 mg of 26 is obtained as a colorless, amorphous solid.

### EXAMPLE 9

11β-[4-[7-(N-Isopropyl-N-methylamido)-heptyloxy]-phenyl]-3-methoxy-17β-(tetrahydropyran-2-yloxy)-81β-vinyl-estra-1,3,5(10)-triene (27)

150 mg of steroid 10 in the reaction with 8-bromo-N-isopropyl-N-methyl-octanamide analogously to instructions 7.1 yields 128 mg of 27 as a colorless foam.

11β-[4-[7-(N-Isopropyl-N-methylamido)-heptyloxy]-phenyl]-8β-vinyl-estra-1,3,5(10)-triene-3,17,β-diol (28)

120 mg of steroid 27 is reacted analogously to instructions 1.5. 54 mg of 28 is obtained as a colorless, amorphous solid.

### EXAMPLE 10

11β-[4-[7-(N-Butyl-N-methylamido)-heptyloxy]-phenyl]-3-methoxy-17β-(tetrahydropyran-2-yloxy)-8β-vinyl-estra-1,3,5(10)-triene (29)

150 mg of steroid 10 in the reaction with 8-bromo-N-butyl-N-methyl-octanamide analogously to instructions 7.1 yields 129 mg of 29 as a colorless foam.

11β-[4-[7-(N-Butyl-N-methylamido)-heptyloxy]-phenyl]-8β-vinyl-estra-1,3,5(10)-triene-3,17β-diol (30)

120 mg of steroid 29 is reacted analogously to instructions 1.5. 56 mg of 30 is obtained as a colorless, amorphous solid.

### EXAMPLE 11

11β-[4-(5-Chloropentyloxy)-phenyl]-3-methoxy-8β-methyl-17β-(tetrahydropyran-2-yloxy)-estra-1,3,5(10)-triene (31)

300 mg of steroid 4 in the reaction with 1-bromo-5-chloropentane analogously to instructions 7.1 yields 249 mg of 31 as a colorless foam.

General Instructions for Introducing Thioacetate in a Two-Stage Procedure [10]

A solution of corresponding steroid [31, 35] in methylethylketone (5 ml/mmol) is mixed with 1.5 equivalents of sodium iodide and refluxed overnight. The solvent is distilled off, the residue is taken up in ethyl acetate, washed with water, dried on magnesium sulfate and concentrated by evaporation again.

The thus obtained viscous yellow oil is dissolved in ethanol (10 ml/mmol) without further purification and mixed with 2 equivalents of potassium thioacetate. The reaction mixture is heated to 50° C. until conversion is completed, mixed with ethyl acetate, washed several times with water and dried on magnesium sulfate. The residue that is obtained after the solvent is removed is purified by column chromatography (cyclohexane/ethyl acetate 3:1).

11β-[4-[5-(S-Acetylthio)-pentyloxy]-phenyl]-3-methoxy-8β-methyl-17β-(tetrahydropyran-2-yloxy)-estra-1,3,5(10)-triene (32)

240 mg of steroid 31 is reacted analogously to instructions 11.1. 187 mg of 32 is obtained as a colorless, viscous oil.

General Operating Instructions for Introducing Pentafluoropentyl Radical [10]

1.5 equivalents of a freshly prepared solution of 5-iodo-1,1,1,2,2-pentafluoropentane [13] and then 2 equivalents of a 10N sodium hydroxide solution are added in drops to a solution of corresponding steroid (32, 36) in tetrahydrofuran/methanol (1:1, 5 ml/mmol). It is heated to 50° C. until conversion is completed, neutralized with 2N hydrochloric acid and extracted several times with dichloromethane. The organic phases are washed several times with water, dried on magnesium sulfate and concentrated by evaporation. The purification of the residue is carried out by column chromatography (cyclohexane/ethyl acetate 5:1).

3-Methoxy-8β-methyl-11β-[4-[5-[(4,4,5,5,5-pentafluoropentyl)-thio]-pentyloxy]-phenyl]-17β-(tetrahydropyran-2-yloxy)-estra-1,3,5(10)-triene (33)

180 mg of steroid 32 is reacted analogously to instructions 11.2 and yields 161 mg of 33 as a colorless foam.

8β-Methyl-11β-[4-[5-[(4,4,5,5,5-pentafluoropentyl)-thio]-pentyloxy]-phenyl]-estra-1,3,5(10)-triene-3,17β-diol (34)

150 mg of steroid 33 is reacted analogously to instructions 1.5. 74 mg of 34 is obtained as a colorless, amorphous solid.

### EXAMPLE 12

11β-[4-(5-Chloropentyloxy)-phenyl]-3-methoxy-17β-(tetrahydropyran-2-yloxy)-8β-vinyl-estra-1,3,5(10)-triene (35)

200 mg of steroid 10 in the reaction with 1-bromo-5-chloropentane analogously to instructions 7.1 yields 158 mg of 35 as a colorless foam.

11β-[4-[5-(S-Acetylthio)-pentyloxy]-phenyl]-3-methoxy-17β-(tetrahydropyran-2-yloxy)-8β-vinyl-estra-1,3,5(10)-triene (36)

155 mg of steroid 35 is reacted analogously to instructions 11.1. 124 mg of 36 is obtained as a colorless, viscous oil.

3-Methoxy-11β-[4-[5-[(4,4,5,5,5-pentafluoropentyl)-thio]-pentyloxy]-phenyl]-17β-(tetrahydropyran-2-yloxy)-8β-vinyl-estra-1,3,5(10)-triene (37)

120 mg of steroid 36 is reacted analogously to instructions 11.2, and 110 mg of 37 is produced as a colorless foam.

11β-[4-[5-[(4,4,5,5,5-Pentafluoropentyl)-thio]-pentyloxy]-phenyl]-8β-vinyl-estra-1,3,5(10)-triene-3,17β-diol (38)

100 mg of steroid 37 is reacted analogously to instructions 1.5. 46 mg of 38 is obtained as a colorless, amorphous solid.

### EXAMPLE 13

General Instructions for Oxidation of a Sulfide with Sodium Metaperiodate [12]

1.6 equivalents of an aqueous 0.5 M sodium metaperiodate solution is added to a solution of corresponding steroid (34, 38) in methanol (40 ml/mmol). The mixture is refluxed until conversion is completed, diluted with water and extracted several times with chloroform. The organic phase is dried with magnesium sulfate and concentrated by evaporation. The purification is carried out by column chromatography (cyclohexane/ethyl acetate 1:2).

8β-Methyl-11β-[4-[5-[(4,4,5,5,5-pentafluoropentyl)-sulfinyl]-pentyloxy]-phenyl]-estra-1,3,5(10)-triene-3,17β-diol (39)

25 mg of steroid 34 is reacted analogously to instructions 13.1. 24 mg of 39 is obtained as a colorless, amorphous solid.

### EXAMPLE 14

11β-[4-[5-[(4,4,5,5,5-Pentafluoropentyl)-sulfinyl]-pentyloxy]-phenyl]-8β-vinyl-estra-1,3,5(10)-triene-3,17β-diol (41)

15 mg of steroid 38 is reacted analogously to instructions 13.1. 14 mg of 41 is obtained as a colorless, amorphous solid.

### EXAMPLE 15

General Instructions for Oxidation of a Sulfide with 3-Chloroperbenzoic Acid [12]

1.2 equivalents of 3-chloroperbenzoic acid (80%) is added at 0° C. to a solution of corresponding steroid (34, 38) in dichloromethane (10 ml/mmol). The mixture is stirred at 0° C. until conversion is completed. For working-up, it is diluted with dichloromethane, washed with saturated sodium thiosulfate solution, saturated sodium bicarbonate solution and water, dried with magnesium sulfate and concentrated by evaporation. The purification is carried out by column chromatography (cyclohexane/ethyl acetate 1:2).

8β-Methyl-11β-[4-[5-[(4,4,5,5,5-pentafluoropentyl)-sulfonyl]-pentyloxy]-phenyl]-estra-1,3,5,(10)-triene-3,17β-diol (40)

25 mg of steroid 34 is reacted analogously to instructions 15.1. 20 mg of 40 is obtained as a colorless, amorphous solid.

### EXAMPLE 16

11β-[4-[5-[(4,4,5,5,5-Pentafuoropentyl)-sulfonyl]-pentyloxy]-phenyl]-8β-vinyl-estra-1,3,5(10)-triene-3,17β-diol (42)

15 mg of steroid 38 is reacted analogously to instructions 15.1. 11 mg of 42 is obtained as a colorless, amorphous solid.

Synthesis of (4-Benzyloxyphenyl)tributyltin (43) that Consists of 4-Bromophenol in 2 Stages [5]

4.87 g of potassium carbonate is added to a solution of 5.54 g of 4-bromophenol and 3.4 ml of benzyl bromide in 160 ml of acetone. The reaction mixture is refluxed for 8 hours. Then, it is filtered, and the filtrate is concentrated by evaporation. The residue is taken up in ethyl acetate, washed several times with 2N sodium hydroxide solution, water and saturated sodium chloride solution, dried on magnesium sulfate and concentrated by evaporation. The colorless solid that is obtained (7.46 g) is used without further purification in the next stage.

The solid in 145 ml of diethyl ether is dissolved. 20 ml of n-butyllithium (1.6 M) is added in drops at −30° C. and stirred for about 2 hours at 0° C. The solution is cooled to −10° C., and 8.7 ml of tributyltin chloride is added in drops to it. It is stirred overnight at room temperature, and the reaction mixture is mixed with saturated ammonium chloride solution/water/diethyl ether (1:1:1). The aqueous phase is extracted several times with diethyl ether, the combined organic phases are washed with water and saturated sodium chloride solution, dried on magnesium sulfate and concentrated by evaporation. Then, the highly volatile components are distilled off in an oil pump vacuum (bath temperature: 200° C.), and the residue is filtered on a flit that is packed with silica gel (0.015-0.04 mm) (mobile solvent: cyclohexane). 6.28 g of aryltributyltin 43 is obtained as a colorless, viscous oil.

Synthesis of ω-Bromo-N,N-dialkylamides (44, 45) [9]

The synthesis of ω-bromo-N,N-dialkylamides (44, 45) is described in the literature [9] and is carried out in very much the same way.

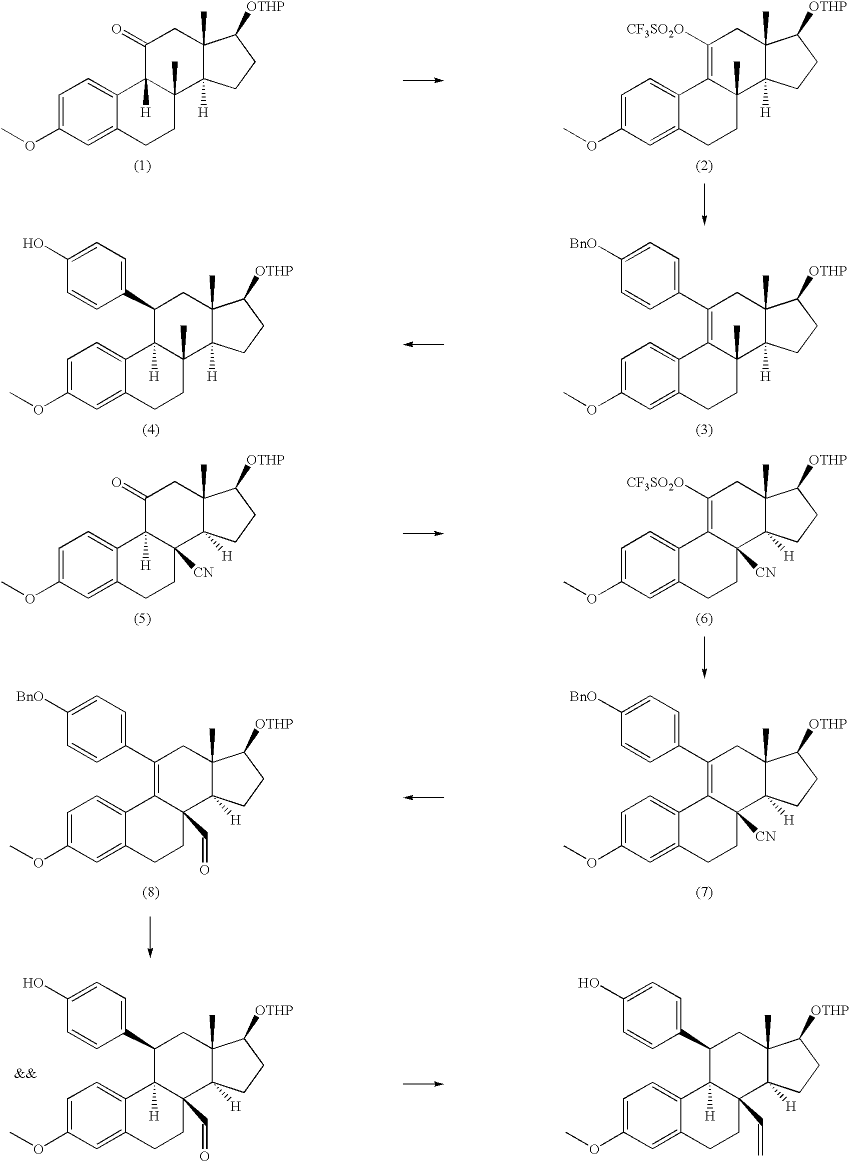

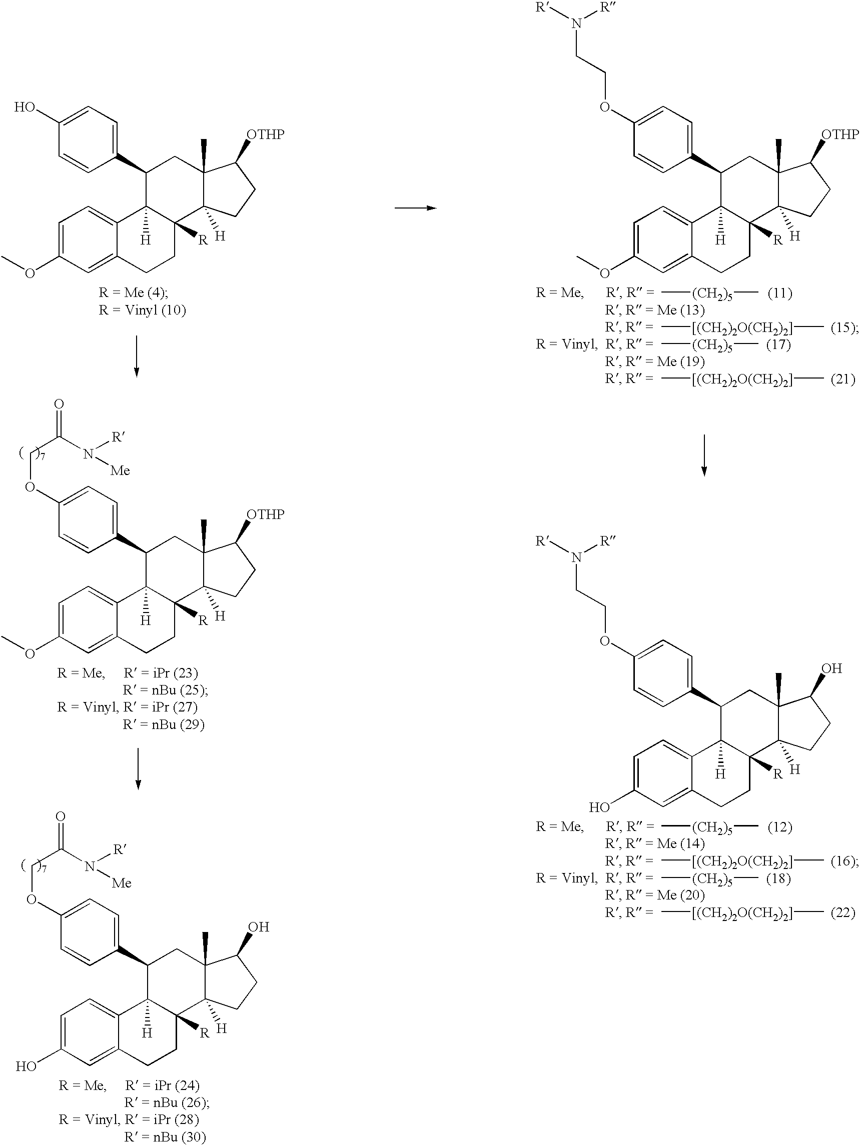

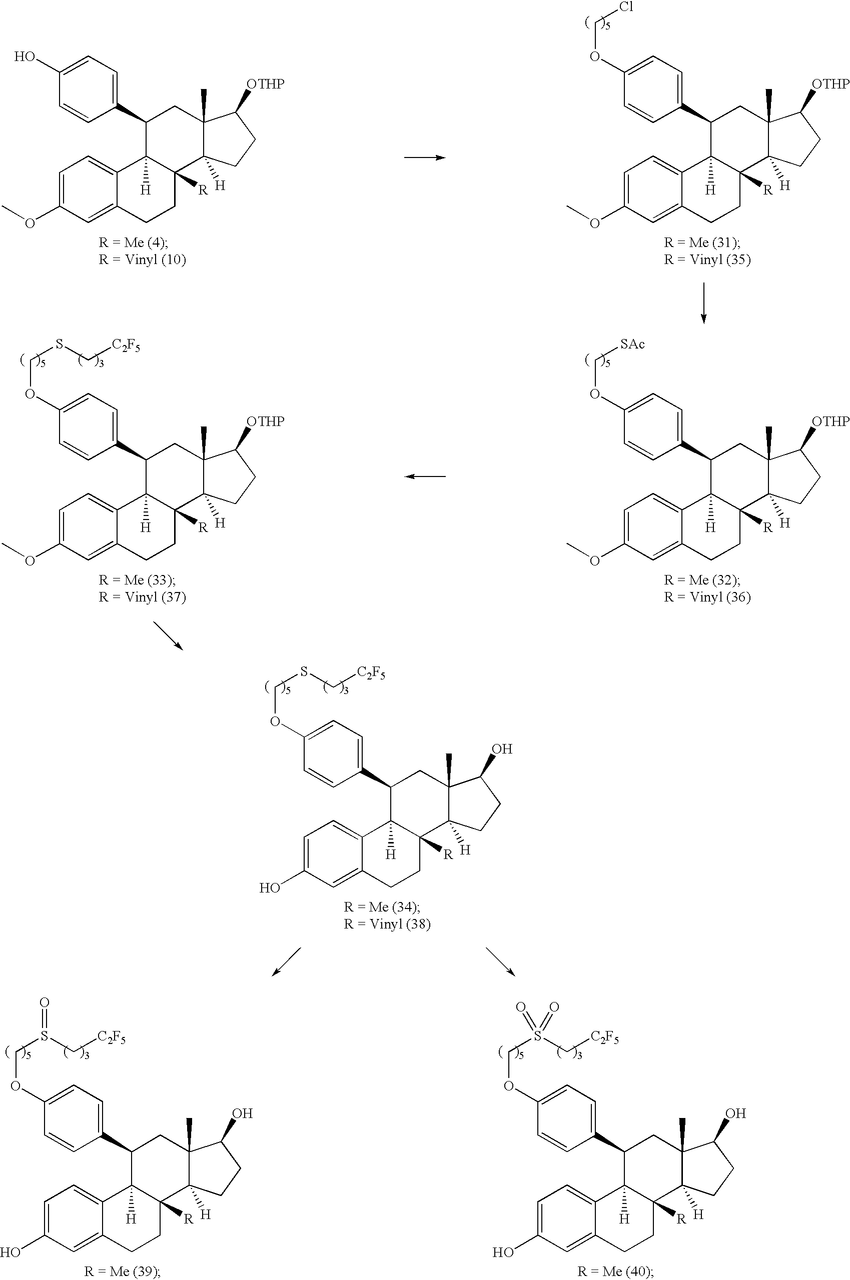

### LITERATURE CONCERNING THE SYNTHESIS OF THE COMPOUNDS ACCORDING TO THE INVENTION

[1] Synthese Steroid [Steroid Synthesis] (1): PCT/EP 01/04289

[2] Synthese Steroid (5): PCT/EP 01/04290

[3] Darstellung Enoltriflate (2, 6) aus 11-Ketonen (1, 5) [Production of Enol Triflates (2, 6) from 11-Ketones (1, 5); e.g, Synthesis (1980), 283; Synthesis (1993), 735

[4] Stille Kupplung der Enoltriflate (2, 6) mit Arylzinn (43) [Stille Coupling of Enol Triflates (2, 6) with Aryl Tin (43): e.g., Tetrahedron Lett. (1991), 32, 4243; J. Org. Chem. (1993), 58, 5434

[5] Darstellung Arylzinn (43) aus dem entsprechenden Arylbromid [Production of Aryl Tin (43) from the Corresponding Aryl Bromide]: J. Org. Chem. (1993), 58, 5434

[6] Synthesesequenz [Synthesis Sequence] (7→10): PCT/EP 01/04289

[7] Einführung Ethanolamin-Seitenketten unter Mitsunobu-Bedingungen [Introduction of Ethanolamine Side Chains under Mitsunobu Conditions] (11, 13, 15, 17, 19, 21): e.g., J. Med. Chem. (1998), 41, 1272;Bioorg. Med. Chem. Lett. (2001), 11, 2521

[8] Einführung der Amid-Seitenketten unter basischen Bedingungen [Introduction of Amide Side Chains under Basic Conditions] (23, 25, 27, 29): e.g., U.S. Pat. No. 5,149,696; J. Steroid Biochem. Molec. Biol. (1994), 50, 21

[9] Synthese Seitenketten [Side-Chain Synthesis] 44, 45: e.g., U.S. Pat. No. 5,149,696

[10] Einführung und Aufbau der Thio-Seitenkette in 33, 37 [Introduction and Build-Up of the Thio Side Chain in 33, 37]: e.g., WO 93/13123; U.S. Pat. No. 6,281,204B1; J. Steroid Biochem. Molec. Biol. (1994), 48, 187

[11] Entschützung 3-Methyl- und 17-THP-Ether [Protection Removal of 3-Methyl Ether and 17-THP-Ether]: e.g., J. Org. Chem. (1999), 64, 9719

[12] Oxidation Sulfid (34, 38) zu Sulfoxid (39, 41) bzw. Sulfon (40, 42) [Oxidation of Sulfide (34, 38) to Sulfoxide (39, 41) or Sulfone (40, 42)]: e.g., J. Steroid Biochem. Molec. Biol. (1994), 50, 21: J. Steroid Biochem. Molec. Biol., (1994), 48, 187

[13] Synthese 4,4,5,5,5-Pentafluoriodpentan [Synthesis of 4,4,5,5,5-Pentafluoroiodopentane]: e.g., U.S. Pat. No. 6,281,204B1

The entire disclosures of all applications, patents and publications, cited herein and of corresponding German Application No. 101 51 114.0, filed Oct. 15, 2001 and U.S. Provisional Application Serial No. 60/330,728, filed Oct. 29, 2001, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula I 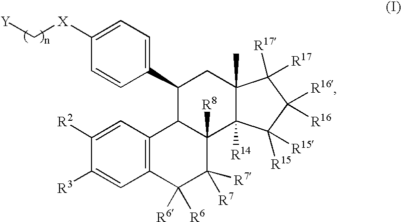
in which
R² means hydrogen, halogen (F, Cl, Br, I);
a radical R¹⁸ or R¹⁸O, whereby R¹⁸ means hydrogen, an alkyl radical or alkanoyl radical (straight-chain or branched-chain, saturated or unsaturated with up to 6 carbon atoms and up to 2 multiple bonds), a benzoyl radical, a trifluoromethyl group;
a radical R¹⁹SO₂O, in which R¹⁹ means an R²⁰R²¹N group, in which R²⁰ and R²¹, independently of one another, mean a hydrogen, a C₁-C₅-alkyl radical, a group C(O)R²², in which R²² means a hydrocarbon radical (optionally substituted, straight-chain or branched-chain, saturated or unsaturated in up to three places, partially or completely halogenated) with up to 10 carbon atoms), an optionally substituted C₃-C₇-cycloalkyl radical, an optionally substituted C₄-C₁₅-cycloalkylalkyl radical or an optionally substituted aryl, heteroaryl or aralkyl radical, or, together with the N-atom, means a polymethylenimino radical with 4 to 6 C atoms or a morpholino radical;
R³ means a radical R¹⁸O, R¹⁹SO₂O or OC(O)R²², with R¹⁸, R¹⁹ and R²² in the meaning that is indicated under R², and in addition R¹⁸ means an aryl, hetaryl or aralkyl radical;
R⁶,R⁷ mean hydrogen;
R^{6′} means hydrogen, a hydroxy group, a group R²² in the meaning that is indicated under R²;
R^{7′} means hydrogen, halogen, a group R¹⁸O, R¹⁹SO₂O, OC(O)R²², with R¹⁸, R¹⁹, and R²² in the meaning that is indicated under R²;
R⁸ means an alkyl radical or alkenyl radical (both straight-chain or branched-chain, optionally partially or completely halogenated, with up to 5 carbon atoms), an ethinyl or prop-1-inyl radical;
X means a direct bond, an oxygen or sulfur atom,
n means an integer from 1 to 12, whereby the corresponding alkylene group can be interrupted by up to 3 O, S or N-methyl,
Y means an amine —NR²⁵R²⁶, with R²⁵ and R²⁶ of the same or a different type, hydrogen, a hydrocarbon radical (optionally substituted, straight-chain or branched-chain, saturated or unsaturated in up to three places, optionally partially or completely halogenated) with up to 10 carbon atoms, an optionally substituted C₃-C₇-cycloalkyl radical, an optionally substituted C₄-C₁₅-cycloalkylalkyl radical or an optionally substituted aryl, heteroaryl or aralkyl radical or together with the nitrogen a heterocyclic compound (optionally with another heteoratom (O, S, N) that is optionally substituted with a C₁-C₄-alkyl radical),
an amide CO—NR²⁵R²⁶ with R²⁵, R²⁶ of the same or a different type, hydrogen, a hydrocarbon radical (optionally substituted, straight-chain or branched-chain, saturated or unsaturated in up to three places, optionally partially or completely halogenated) with up to 10 carbon atoms, an optionally substituted C₃-C₇-cycloalkyl radical, an optionally substituted C₄-C₁₅-cycloalkylalkyl radical or an optionally substituted aryl, heteroaryl or aralkyl radical or together with the nitrogen a heterocyclic compound (optionally with another heteroatom (O, S, N), optionally substituted with a C₁-C₄-alkyl radical),
a grouping —S(O)ₘR²⁷ with m=0, 1 or 2, and R²⁷ as a hydrocarbon radical (optionally substituted, straight-chain or branched-chain, saturated or unsaturated in up to three places, optionally partially or completely halogenated) with up to 10 carbon atoms, an optionally substituted C₃-C₇-cycloalkyl radical; an optionally substituted C₄-C₁₅-cycloalkylalkyl radical or an optionally substituted aryl, heteroaryl or aralkyl radical;
R¹⁴ means hydrogen, an additional bond with R¹⁶;
R¹⁵ means hydrogen, an additional bond with R¹⁴, R⁶;
R¹⁶ means hydrogen, a bond with R¹⁵;
R^{15′},R^{16′}, independently of one another, mean hydrogen, halogen, a group R¹⁸O, R¹⁹SO₂O or OC(O)R²², with R¹⁸, R¹⁹ and R²² in each case in the meaning that is indicated under R²;
R¹⁷, R^{17′} each mean a hydrogen atom;
a hydrogen atom and a halogen atom;
a hydrogen atom and a benzyloxy group;
a hydrogen atom and a group R¹⁹SO₂—O—;
a group R¹⁸ and a group —C(O)R²² or —O—C(O)R²²;
a group R¹⁸ —O— and a group R¹⁸—;
a group R¹⁸ —O— and a group —O—C(O)R²², in each case with R¹⁸, R¹⁹ and R²² in the meaning that is indicated under R²;
R¹⁷, R^{17′} together mean a group ═CR²³R²⁴, in which R²³ and R²⁴, independently of one another, represent a hydrogen atom and a halogen atom, or together represent an oxygen atom, as well as their pharmacologically compatible salts with acids in the case of the presence of a basic nitrogen atom in the compound of general formula I.

2. Compounds of general formula I according to claim 1, in which
R6, R6′, R7, R7′, R14, R15 and R15′ in each case mean a hydrogen atom, and
X means an oxygen or sulfur atom.

3. Compounds according to claim 2, in which X stands for an oxygen atom.

4. Compounds according to claim 1, in which R⁸ stands for a methyl group.

5. Compounds according to claim 1, in which R⁸ stands for a vinyl group.

6. Compounds according to claim 1, in which R⁸ stands for an ethinyl group.

7. Compounds according to claim 1, in which R³ stands for a hydroxy group.

8. Compounds according to claim 1, in which R¹⁷, R¹⁷ stand for a hydrogen atom and a hydroxy group.

9. Compounds according to claim 8, in which R³ stands for a hydroxy group.

10. Compounds according to claim 1, in which R³ stands for a group R¹⁹SO₂O.

11. Compounds according to claim 1, in which R¹⁷, R^{17′} stand for a hydrogen atom and a group R¹⁹SO₂—O—.

12. Compounds according to claim 1, in which R3 stands for a group —OC(O)R²².

13. Compounds according to claim 1, in which R¹⁷, R^{17′} stand for a hydrogen atom and a group —O—C(O)R²².

14. Compounds according to claim 1, in which substituent —X—(—)ₙ—Y is selected from the group of substituents 2-(dimethylamino)ethoxy, 2-(N-methyl-N-phenyl-amino)ethoxy, 2-(1-piperidinyl)ethoxy, 2-(1-pyrrolidinyl)ethoxy, 2-(1-morpholinyl)ethoxy, N-butyl-N-methyl-8-octanamidoyl, N-isopropyl-N-methyl-8-octanamidoyl, and 5-[(4,4,5,5,5-pentafluoropentyl)sulfonyl]pentyloxy.

15. Compounds of the general formula according to claim 1, namely
8β-Methyl-11β-[4-(2-N-piperidinyl-ethoxy)-phenyl]-estra-1,3,5(10)-triene-3,17β-diol
11β-[4-(N,N,-dimethyl-ethoxy)-phenyl]-8β-methyl-estra-1,3,5(10)-triene-3,17β-diol
8β-methyl-11β-[4-(2-N-morpholinyl-ethoxy)-phenyl]-estra-1,3,5(10)-triene-3,17β-diol
8β-methyl-11β-[4-[2-(1-pyrrolindinyl)ethoxy]-phenyl]-estra-1,3,5(10)-triene-3,17β-diol
8β-methyl-11β-[4-[2-(N-methyl-N-phenyl-amino)ethoxy]-estra-1,3,5(10)-triene-3,17β-diol
8β-methyl-11β-[4-(2-N-piperidinyl-ethoxy)-phenyl]-estra-1,3,5(10)-triene-3,17β-diol-3-sulfamate
11β-[4-(N,N,-dimethyl-ethoxy)-phenyl]-8β-methyl-estra-1,3,5(10)-triene-3,17β-diol-3-sulfamate
8β-methyl-11β-[4-(2-N-morpholinyl-ethoxy)-phenyl]-estra-1,3,5(10)-triene-3,17β-diol-3-sulfamate
8β-methyl-11β-[4-[2-(1-pyrrolidinyl)ethoxy]phenyl]-estra-1,3,5(10)-triene-3,17β-diol-3-sulfamate
8β-methyl-11β-[4-[2-(N-methyl-N-phenyl-amino)ethoxy]-estra-1,3,5(10)-triene-3,17β-diol-3-sulfamate
8β-methyl-11β-[4-(2-N-piperidinyl-ethoxy)-phenyl]-estra-1,3,5(10)-triene-3,17β-diol-3-acetate
11β-[4-(N,N,-dimethyl-ethoxy)-phenyl]-8β-methyl-estra-1,3,5(10)-triene-3,17β-diol-3-acetate
8β-methyl-11β-[4-(2-N-morpholinyl-ethoxy)-phenyl]-estra-1,3,5(10)-triene-3,17β-diol-3-acetate
8β-methyl-11β-[4-[2-(1-pyrrolidinyl)ethoxy]phenyl]-estra-1,3,5(10)-triene-3,17β-diol-3-acetate
8β-methyl-11β-[4-[2-(N-methyl-N-phenyl-amino)ethoxy]-estra-1,3,5(10)-triene-3,17β-diol-3-acetate
11β-[4-(2-N-piperidinyl-ethoxy)-phenyl]-8β-vinyl-estra-1,3,5(10)-triene-3,17β-diol
11β-[4-(N,N,-dimethyl-ethoxy)-phenyl]-8β-vinyl-estra-1,3,5(10)-triene-3,17β-diol
11β-[4-(2-N-morpholinyl-ethoxy)-phenyl]-8β-vinyl-estra-1,3,5(10)-triene-3,17β-diol
11β-[4-[2-(1-pyrrolidinyl)ethoxy]-8β-vinyl-estra-1,3,5(10)-triene-3,17β-diol
11β-[2-(N-methyl-N-phenyl-amino)ethoxy]-8β-vinyl-estra-1,3,5(10)-triene-3,17β-diol
11β-[4-(2-N-piperidinyl-ethoxy)-phenyl]-8β-vinyl-estra-1,3,5(10)-triene-3,17β-diol-3-sulfamate
11β-[4-(N,N,-dimethyl-ethoxy)-phenyl]-8β-vinyl-estra-1,3,5(10)-triene-3,17β-diol-3-sulfamate
11β-[4-(2-N-morpholinyl-ethoxy)-phenyl]-8β-vinyl-estra-1,3,5(10)-triene-3,17β-diol-3-sulfamate
11β-[4-[2-(1-pyrrolidinyl)ethoxy]-8β-vinyl-estra-1,3,5(10)-triene-3,17β-diol-3-sulfamate
11β-[2-(N-methyl-N-phenyl-amino)ethoxy]-8β-vinyl-estra-1,3,5(10)-triene-3,17β-diol-3-sulfamate
11β-[4-(2-N-piperidinyl-ethoxy)-phenyl]-8β-vinyl-estra-1,3,5(10)-triene-3,17β-diol-3-acetate
11β-[4-(N,N,-dimethyl-ethoxy)-phenyl]-8β-vinyl-estra-1,3,5(10)-triene-3,17β-diol-3-acetate
11β-[4-(2-N-morpholinyl-ethoxy)-phenyl]-8β-vinyl-estra-1,3,5(10)-triene-3,17β-diol-3-acetate
11β-[4-[2-(1-pyrrolidinyl)ethoxy]-8β-vinyl-estra-1,3,5(10)-triene-3,17β-diol-3-acetate
11β-[2-(N-methyl-N-phenyl-amino)ethoxy]-8β-vinyl-estra-1,3,5(10)-triene-3,17-diol-3-acetate
11β-[4-[7-(N-isopropyl-N-methylamido)-heptyloxy]-phenyl]-8β-methyl-estra-1,3,5(10)-triene-3,17β-diol
11β-[4-[7-(N-butyl-N-methylamido)-heptyloxy]-pheny]-8β-methyl-estra-1,3,5(10)-triene-3,17β-diol
11β-[4-[7-(N-isopropyl-N-methylamido)-heptyloxy]-phenyl]-8β-vinyl-estra-1,3,5(10)-triene-3,17β-diol
11β-[4-[7-(N-butyl-N-methylamido)-heptyloxy]-phenyl]-8β-vinyl-estra-1,3,5(10)-triene-3,17β-diol
11β-[4-[7-(N-isopropyl-N-methylamido)-heptyloxy]-phenyl]-8β-methyl-estra-1,3,5(10)-triene-3,17β-diol-3-sulfamate
11β-[4-[7-(N-butyl-N-methylamido)-heptyloxy]-phenyl]-8β-methyl-estra-1,3,5(10)-triene-3,17β-diol-3-sulfamate
11β-[4-[7-(N-isopropyl-N-methylamido)-heptyloxy]-phenyl]-8β-vinyl-estra-1,3,5(10)-triene-3,17β-diol-3-sulfamate
11β-[4-[7-(N-butyl-N-methylamido)-heptyloxy]-phenyl]-8β-vinyl-estra-1,3,5(10)-triene-3,17β-diol-3-sulfamate
11β-[4-[7-(N-isopropyl-N-methylamido)-heptyloxy]-phenyl]-8β-methyl-estra-1,3,5(10)-triene-3,17β-diol-3-acetate
11β-[4-[7-(N-butyl-N-methylamido)-heptyloxy]-phenyl]-8β-methyl-estra-1,3,5(10)-triene-3,17β-diol-3-acetate
11β-[4-[7-(N-isopropyl-N-methylamido)-heptyloxy]-phenyl]-8β-vinyl-estra-1,3,5(10)-triene-3,17β-diol-3-acetate
11β-[4-[7-(N-butyl-N-methylamido)-heptyloxy]-phenyl]-8β-vinyl-estra-1,3,5(10)-triene-3,17β-diol-3-acetate
8β-methyl-11β-[4-[5-[(4,4,5,5,5-pentafluoropentyl)-thio]-pentyloxy]-phenyl]-estra-1,3,5(10)-triene-3,17β-diol
11β-[4-[5-[(4,4,5,5,5-pentafluoropentyl)-thio]-pentyloxy]-phenyl]-8β-vinyl-estra-1,3,5(10)-triene-3,17β-diol
8β-methyl-11β-[4-[5-[(4,4,5,5,5-pentafluoropentyl)-sulfinyl]-pentyloxy]-phenyl]-estra-1,3,5(10)-triene-3,17β-diol
11β-[4-[5-[(4,4,5,5,5-pentafluoropentyl)-sulfinyl]-pentyloxy]-phenyl]-8β-vinyl-estra-1,3,5(10)-triene-3,17β-diol
8β-methyl-11β-[4-[5-[(4,4,5,5,5-pentafluoropentyl)-sulfonyl]-pentyloxy]-phenyl]-estra-1,3,5(10)-triene-3,17β-diol
11β-[4-[5-[(4,4,5,5,5-pentafluoropentyl)-sulfonyl]-pentyloxy]-phenyl]-8β-vinyl-estra-1,3,5(10)-triene-3,17β-diol.

16. Use of 8β,11β-disubstituted estra-1,3,5(10)-trien derivatives of general formula I according to claim 1 for the production of pharmaceutical agents for contraception in women.

17. Use of 8β,11β-disubstituted estra-1,3,5(10)-triene derivatives of general formula I according to claim 1 for the production of pharmaceutical agents for contraception in men.

18. Use of the estratriene derivatives of general formula I according to claim 1 for the production of pharmaceutical agents for treating benign or malignant proliferative diseases of the ovary.

19. Use according to claim 18 for treating ovarian cancer.

20. Use according to claim 18 for treating granulosa cell tumors.

21. Use of the structural portion of 11β-(para-substituted)phenyl-estra-1,3,5(10)-triene with a straight-chain or branched-chain, optionally partially or completely halogenated alkyl radical or alkenyl radical in each case with up to 5 carbon atoms, an ethinyl radical or prop-1-inyl radical in 8β-position as a component of the entire structure of the compounds that have a contraceptive effect on men and women without influencing other estrogen-sensitive organs such as the uterus or the liver.

22. Use of the structural portion of the 11β-(para-substituted)phenyl-estra-1,3,5(10)-triene with a straight-chain or branched-chain, optionally partially or completely halogenated alkyl radical or alkenyl radical in each case with up to 5 carbon atoms, an ethinyl or prop-1-inyl radical in 8β-position as a component of the entire structure of compounds that are suitable for treating benign or malignant proliferative diseases of the ovary, such as ovarian cancer and granulosa cell tumors.

23. Pharmaceutical compositions that contain at least one compound according to one of claims 1 to 15, as well as a pharmaceutically compatible vehicle.

24. Pharmaceutical compositions according to claim 23, which in addition to at least one compound of general formula I according to claim 1 contain at least one compound that is selected from the group of GnRH antagonists, progesterone receptor antagonists, mesoprogestins, gestagens or tissue-selective gestagens.
